(19) United States
(12) Patent Application Publication
(10) Pub. No.: US 2026/0327705 A1
(43) Pub. Date: Oct. 22, 2026

(54) ANALYTIC METHOD FOR DIAGNOSING OVARIAN CANCER RESISTANT TO CISPLATIN

(71) Applicant: CHA UNIVERSITY INDUSTRY-ACADEMIC COOPERATION FOUNDATION, Pocheon-si, Gyeonggi-do (KR)

(72) Inventors: Kwang-Hyun BAEK, Seoul (KR); Hee-Gyeong KO, Gunpo-si, Gyeonggi-do (KR); Ye-Won KIM, Suwon-si, Gyeonggi-do (KR)

(73) Assignee: CHA UNIVERSITY INDUSTRY-ACADEMIC COOPERATION FOUNDATION, Pocheon-si, Gyeonggi-do (KR)

(21) Appl. No.: 19/099,385

(22) PCT Filed: Jul. 20, 2023
(86) PCT No.: PCT/KR2023/010466

§ 371 Date: Apr. 9, 2025

(30) Foreign Application Priority Data
Jul. 29, 2022 (KR) 10-2022-0094364

(51) Int. Cl. C12Q 1/6886; C12Q 1/6837; C12Q 1/6851

(52) U.S. Cl. CPC C12Q 1/6886; C12Q 1/6837; C12Q 1/6851; C12Q 2600/106; C12Q 2600/158

(57) ABSTRACT

Provided are an analytic method to provide information needed for the diagnosis of patients with ovarian cancer resistant to cisplatin which is an anticancer drug widely used in the treatment of ovarian cancer, the method comprising a step of measuring the expression level of a gene encoding at least one deubiquitinating enzyme selected from the group consisting of USP12, USP17, USP51, OTUD1, and PSMD14, in a tumor cell sample externally discharged from an ovarian cancer patient; and a kit for diagnosing ovarian cancer resistant to cisplatin, the kit comprising a molecule capable of measuring the expression level of a gene encoding the protein.

### TECHNICAL FIELD

The present invention relates to an analytical method and a kit for providing information necessary for diagnosing an ovarian cancer patient resistant to cisplatin. More specifically, the present invention relates to an analytical method for providing information necessary for diagnosing an ovarian cancer patient resistant to cisplatin, comprising measuring an expression level of a gene encoding a deubiquitinating enzyme and a kit useful for the analytical method.

### BACKGROUND ART

Ovarian cancer, the second most common gynecological cancer, has the highest mortality rate among all female genital cancers and the incidence thereof is increasing worldwide. Despite the discoveries of diagnostic, surgical techniques and new therapeutic agents, the prognosis of ovarian cancer patients remains poor, which is originated from late diagnosis and the lack of effective treatment methods for resistant disease.

Cisplatin having excellent anticancer effects against ovarian cancer is one of the most commonly used chemotherapy drugs (Song et al, Therapeutic strategies to overcome cisplatin resistance in ovarian cancer, Eur J Med Chem (2022): 114205). However, despite good initial response rates, most patients receiving cisplatin treatment have resistance through various mechanisms (Galluzzi et al, Molecular mechanisms of cisplatin resistance, Oncogene 31, no. 15 (2012): 1869-1883). Although 70% of ovarian cancer patients respond sensitively and are treated by the excellent anticancer effects of cisplatin, half of them develop cisplatin resistance, leading to failure of treatment, fatal recurrence and metastasis, and low survival rates (Yang et al, Exploring cisplatin resistance in ovarian cancer through integrated bioinformatics approach and overcoming chemoresistance with sanguinarine, Am J Transl Res 12, no. 3 (2020): 923).

Therefore, the development of a biomarker capable of confirming ovarian cancer patients resistant to cisplatin in advance makes it possible to increase treatment efficiency by providing appropriate treatment for ovarian cancer patients. In particular, the preemptive confirmation of the presence of resistance through the analysis of such a biomarker can contribute to shortening the treatment time and suggesting an optimal treatment strategy, as well as to the development of a resistance-prediction kit for an anticancer drug against ovarian cancer.

### DETAILED DESCRIPTION OF THE INVENTION

### Technical Problem

In order to identify a biomarker capable of diagnosing ovarian cancer patients resistant to cisplatin, the present inventors analyzed the genes of deubiquitinating enzymes showing the difference in expressions between the ovarian cancer cells having resistance thereto and the ovarian cancer cells not having resistance thereto, through multiplex RT-PCR analysis. As the results thereof, the present inventors found that the genes encoding five deubiquitinating enzymes, which had not been reported as deubiquitinating enzymes associated with ovarian cancer resistant to cisplatin, showed significantly different expression levels in the ovarian cancer resistant to cisplatin, and the results were verified through qRT-PCR. Therefore, the genes encoding these deubiquitinating enzymes may be useful for diagnosing ovarian cancer resistant to cisplatin, and thus said genes may be used as a biomarker for diagnosing ovarian cancer resistant to cisplatin.

Accordingly, it is an object of the present invention to provide an analytical method for providing information necessary for diagnosing an ovarian cancer patient resistant to cisplatin, using the genes encoding the specific deubiquitinating enzymes.

In addition, it is another object of the present invention to provide a kit for diagnosing an ovarian cancer patient resistant to cisplatin, comprising a molecule capable of measuring an expression level of the genes encoding said deubiquitinating enzymes.

### Technical Solution

In accordance with an aspect of the present invention, there is provided an analytical method for providing information necessary for diagnosing an ovarian cancer patient resistant to cisplatin, comprising measuring an expression level of a gene encoding at least one deubiquitinating enzyme selected from the group consisting of USP12, USP17, USP51, OTUD1, and PSMD14 in a tumor cell sample externally discharged from an ovarian cancer patient.

The measuring an expression level of the gene encoding the deubiquitinating enzyme may be carried out by measuring an amount of mRNA of the gene. For example, the measuring an amount of mRNA may be carried out by RT-PCR or qRT-PCR.

In an embodiment, the analytical method of the present invention may comprise measuring an expression level of the gene encoding at least one deubiquitinating enzyme selected from the group consisting of USP12 and USP51. In said embodiment, the measuring an expression level of the gene may be carried out by measuring an amount of mRNA of the gene with a primer set of SEQ ID NOs: 1 and 2 or a primer set of SEQ ID NOs: 7 and 8.

In another embodiment, the analytical method of the present invention may comprise measuring an expression level of the gene encoding at least one deubiquitinating enzyme selected from the group consisting of USP17, OTUD1, and PSMD14. In said embodiment, the measuring an expression level of the gene may be carried out by measuring an amount of mRNA of the gene with a primer set of SEQ ID NOs: 3 and 4, a primer set of SEQ ID NOs: 9 and 10, or a primer set of SEQ ID NOs: 11 and 12.

In accordance with another aspect of the present invention, there is provided a kit for diagnosing an ovarian cancer patient resistant to cisplatin, comprising a molecule capable of measuring an expression level of a gene encoding at least one deubiquitinating enzyme selected from the group consisting of USP12, USP17, USP51, OTUD1, and PSMD14, wherein the molecule is a primer having a complementary sequence specific to the gene encoding the deubiquitinating enzyme.

In the kit of the present invention, the primer may have at least one base sequence selected from the group consisting of SEQ ID NOs: 1 to 4 and 7 to 12. In addition, the kit of the present invention may be in the form of a microarray in which the primer is immobilized on a substrate.

### Advantageous Effects

It has been found by the present invention that the genes encoding specific deubiquitinating enzymes, i.e., the genes encoding USP12, USP17, USP51, OTUD1 or PSMD14, show significantly different expression levels in the ovarian cancer resistant to cisplatin. Therefore, the analytical method and kit according to the present invention, using said genes as a biomarker, can be usefully applied for diagnosing an ovarian cancer patient resistant to cisplatin.

### BRIEF DESCRIPTION OF THE DRAWINGS

FIG. 1 shows the results obtained by measuring the cell viabilities using CCK-8 after treating the ovarian cancer cell line A2780 and the cisplatin-resistant ovarian cancer cell line A2780/cisR with cisplatin (30 μM).

FIGS. 2a to 2c show the results obtained by measuring the mRNA expression levels of the deubiquitinating enzymes decreased according to cisplatin resistance in the ovarian cancer cell line A2780, through Multiplex RT-PCR (A: A2780 cells, B: A2780/cisR cells).

FIGS. 3a to 3c show the results obtained by measuring the mRNA expression levels of the deubiquitinating enzymes increased according to cisplatin resistance in the ovarian cancer cell line A2780, through Multiplex RT-PCR (A: A2780 cells, B: A2780/cisR cells).

FIG. 4 shows the statistical analysis results of the relative mRNA expression levels of the deubiquitinating enzymes USP12, USP46, and USP51 from the results of FIG. 2. (***: p<0.001, **: 0.001<p<0.01, *: 0.01<p<0.05, ns: p>0.05)

FIG. 5 shows the statistical analysis results of the relative mRNA expression levels of the deubiquitinating enzymes USP17, OTUD1, and PSMD14 from the results of FIG. 3. (***: p<0.001, **: 0.001<p<0.01, *: 0.01<p<0.05, ns: p>0.05)

FIG. 6 shows the statistical analysis results of the relative mRNA expression levels of the deubiquitinating enzymes USP12, USP46, and USP51, which were decreased in the ovarian cancer cell line A2780/cisR compared to the ovarian cancer cell line A2780, through qRT-PCR. (***: p<0.001, **: 0.001<p<0.01, *: 0.01<p<0.05, ns: p>0.05)

FIG. 7 shows the statistical analysis results of the relative mRNA expression levels of the deubiquitinating enzymes USP17, OTUD1, and PSMD14, which were increased in the ovarian cancer cell line A2780/cisR compared to the ovarian cancer cell line A2780, through qRT-PCR. (***: p<0.001, **: 0.001<p<0.01, *: 0.01<p<0.05, ns: p>0.05)

### BEST MODE FOR CARRYING OUT THE INVENTION

As used herein, the term “a tumor cell sample externally discharged from an ovarian cancer patient” refers to a cell or tissue sample externally discharged from ovarian cancer patient's tumor cells through a biopsy or the like. In hospitals, ovarian cancer tumor cells and tissues are usually collected from patients and various tests are carried out, in order to diagnose and establish treatment plans for ovarian cancer patients. Therefore, as used herein, the term “a tumor cell sample externally discharged from an ovarian cancer patient” refers to a cell or tissue sample externally discharged from the patient for tissue examination or the like at a hospital.

Using the primer sets for genes encoding deubiquitinating enzymes produced in our laboratory (including e.g., the primer sets of Korean Patent Publication No. 10-2018-0050098), the present inventors identified the genes of deubiquitinating enzymes that specifically show different expression levels in the ovarian cancer cells having cisplatin resistance, through multiplex polymerase chain reaction (multiplex RT-PCR). From the results thereof, on the genes encoding the specifically under-expressed or over-expressed deubiquitinating enzymes in the cisplatin-resistant ovarian cancer cell line A2780/cisR compared to the control group, i.e., the genes of USP12, USP17, USP46, USP51, OTUD1, and PSMD14, we additionally carried out qRT-PCR to quantify and verify the differences in mRNA expression levels. Through said Multiplex RT-PCR and qRT-PCR analyses, it was found that the mRNA expressions of USP12, USP46, and USP51 were down-regulated in the cisplatin-resistant ovarian cancer cell line (A2780/cisR), whereas the mRNA expressions of USP17, OTUD1, and PSMD14 were up-regulated in the cisplatin-resistant ovarian cancer cell line (A2780/cisR). In addition to USP46 which was found to be differentially expressed in the previous study performed on cisplatin-resistant ovarian cancer cells, USP12, USP17, USP51, OTUD1, and PSMD14 may serve as a biomarker for diagnosing cisplatin resistance in ovarian cancer treatment.

The present invention provides an analytical method for providing information necessary for diagnosing an ovarian cancer patient resistant to cisplatin, comprising measuring an expression level of a gene encoding at least one deubiquitinating enzyme selected from the group consisting of USP12, USP17, USP51, OTUD1, and PSMD14 in a tumor cell sample externally discharged from an ovarian cancer patient.

Both the protein sequences and the base sequences of the genes encoding the deubiquitinating enzymes USP12, USP17, USP51, OTUD1, and PSMD14 used as a biomarker in the analysis method of the present invention are known in the art, and therefore the known protein and gene sequences can be used in the analysis method of the present invention. The NCBI accession numbers of the USP12 (ubiquitin-specific peptidase 12) protein are AAH26072.1, AAC23551.1, and BAF82374.1 and the NCBI accession numbers of the mRNA encoding the same are BC026072.1, AF022789.1, AK289685.1, AL049221.1, and so on. The NCBI accession number of the USP17 (ubiquitin-specific peptidase 17) protein is AY509884.1 and the NCBI accession number of the mRNA encoding the same is AAR91701.1. The NCBI accession numbers of the USP51 (ubiquitin-specific peptidase 51) protein are CAE47750.2, AAH35907.1, and CAE48396.2 and the NCBI accession numbers of the mRNA encoding the same are AI934832.1, AJ583823.2, BC035907.1, BC131541.1, BN000340.2, BU101641.1, and so on. The NCBI accession numbers of the mRNA encoding the OTUD1 (ovarian tumor deubiquitinase 1) protein are AB188491.1, AI261652.1, AK096389.1, and so on. The NCBI accession numbers of the PSMD14 (proteasome 26S subunit, non-ATPase 14) protein are BAD92457.1, BAG51474.1, AAH09524.1, AAH66336.1, AEE61289.1, and AAC51866.1 and the NCBI accession numbers of the mRNA encoding the same are AB209220.1, AK055128.1, AK091095.1, BC009524.1, BC066336.1, BM978668.1, DA133201.1, HM005692.1, U86782.1, and so on.

The measuring an expression level of the genes encoding the deubiquitinating enzymes may be carried out according to a method conventionally used in the field of biotechnology. For example, the measuring an expression level of the genes encoding the deubiquitinating enzymes may be carried out by measuring the mRNA amount of the genes, and the measuring an amount of mRNA may be carried out by a method such as reverse transcription PCR (RT-PCR) or quantitative real time PCR (qRT-PCR).

In an embodiment, the analytical method of the present invention may comprise measuring an expression level of the gene encoding at least one deubiquitinating enzyme selected from the group consisting of USP12 and USP51. In said embodiment, the measuring an expression level of the gene may be carried out by measuring an amount of mRNA of the gene with a primer set of SEQ ID NOs: 1 and 2 or a primer set of SEQ ID NOs: 7 and 8.

For example, the expression levels of USP12 and/or USP51 are measured though a qRT-PCR method in cells that are not resistant to cisplatin [e.g., A2780 (93112519, Sigma-Aldrich, St. Louis, MO, USA), etc.] and in a tumor cell sample externally discharged from an ovarian cancer patient, respectively; and if the expression levels of USP12 and/or USP51 in the tumor cell sample externally discharged from an ovarian cancer patient is/are significantly (e.g., twice or more) lower than the expression levels of USP12 and/or USP51 in the cells that are not resistant to cisplatin, based on the mRNA expression levels analyzed by the 2^{-ΔΔCt} method through qRT-PCR, the ovarian cancer patient can be classified as an ovarian cancer patient resistant to cisplatin.

In another embodiment, the analytical method of the present invention may comprise measuring an expression level of the gene encoding at least one deubiquitinating enzyme selected from the group consisting of USP17, OTUD1, and PSMD14. In said embodiment, the measuring an expression level of the gene may be carried out by measuring an amount of mRNA of the gene with a primer set of SEQ ID NOs: 3 and 4, a primer set of SEQ ID NOs: 9 and 10, or a primer set of SEQ ID NOs: 11 and 12.

For example, the expression levels of USP17, OTUD1, and/or PSMD14 are measured though a qRT-PCR method in cells that are not resistant to cisplatin [e.g., A2780 (93112519, Sigma-Aldrich, St. Louis, MO, USA), etc.] and in a tumor cell sample externally discharged from an ovarian cancer patient, respectively; and if the expression levels of USP17, OTUD1, and/or PSMD14 in the tumor cell sample externally discharged from an ovarian cancer patient is/are significantly (e.g., twice or more) higher than the expression levels of USP17, OTUD1, and/or PSMD14 in the cells that are not resistant to cisplatin, based on the mRNA expression levels analyzed by the 2^{-ΔΔCt} method through qRT-PCR, the ovarian cancer patient can be classified as an ovarian cancer patient resistant to cisplatin.

The present invention also provides a kit for diagnosing an ovarian cancer patient resistant to cisplatin, comprising a molecule capable of measuring an expression level of a gene encoding at least one deubiquitinating enzyme selected from the group consisting of USP12, USP17, USP51, OTUD1, and PSMD14, wherein the molecule is a primer having a complementary sequence specific to the gene encoding the deubiquitinating enzyme.

In the kit of the present invention, the primer having a complementary sequence specific to the gene encoding the deubiquitinating enzyme may be prepared according to a method conventionally used in the field of biotechnology, and a diagnostic kit comprising the primer may be also prepared. For example, the primer may have at least one base sequence selected from the group consisting of SEQ ID NOs: 1 to 4 and 7 to 12. And, the diagnostic kit of the present invention may be in the form of a microarray, e.g., in the form of a chip such as a DNA chip or a protein chip, in which the primer is immobilized on a substrate.

Hereinafter, the present invention will be described more specifically by the following examples. However, the following examples are provided only for illustrations and thus the present invention is not limited to or by them.

### EXAMPLES

### 1. Test Methods

### (1) Culture of Ovarian Cancer Cell Line and Cisplatin-Resistant Cell Line

Ovarian cancer cell line A2780 (93112519, Sigma-Aldrich, St. Louis, MO, USA) and cisplatin-resistant ovarian cancer cell line A2780/cisR (93112517, Sigma-Aldrich, St. Louis, MO, US) were cultured in RPMI 1640 medium (31800-022, Gibco, Grand Island, NY, USA) containing 10% FBS and 1% Antibiotic-Antimycotic (15240062, Gibco, Grand Island, NY, USA) at 37° C. in a 5% CO₂ incubator.

### (2) Measurement of Cell Viability

After measuring the number of cells, the cells were cultured in a 96-well plate for 24 hours under the condition of 37° C. in a 5% CO₂ incubator so that 5×10³ cells of A2780 and A2780/cisR were grown respectively. The next day, these cells were cultured in RPMI 1640 medium containing 30 μM cisplatin for 0 hour, 24 hours, and 48 hours. CCK-8 solution (Cell Counting Kit-8, CK04-11, Dojindo Molecular Technologies Inc., Japan) was added 1:10 to each culture medium of A2780 and A2780/cisR cultured in RPMI 1640 medium containing 30 μM cisplatin. The cells were cultured for additional 2 hours according to the manufacturer's protocol and then the cell viabilities were confirmed by measuring the absorbances at 450 nm.

(3) RNA Extraction and cDNA Synthesis

The ovarian cancer cell line A2780 and the cisplatin-resistant ovarian cancer cell line A2780/cisR cultured in RPMI 1640 medium were harvested, respectively, and the respective RNA was extracted from the A2780 and A2780/cisR cells using a Trizol solution (15596018, Thermo Fisher Scientific, Waltham, MA, USA). The RNA extractions were confirmed through the 18S rRNA and 28S rRNA bands observed on gel electrophoresis. After RNA quantification, cDNA was synthesized using a cDNA synthesis kit (CMRTK002, Cosmogenetech, Seoul, Korea) with a concentration of 1 μg of each RNA and then the following multiplex polymerase chain reaction (Multiplex RT-PCR) and qRT-PCR were performed.

### (4) Multiplex RT-PCR

Multiplex RT-PCR was carried out using the primers (SEQ ID NOs: 13 and 14) capable of amplifying the housekeeping gene GAPDH and the primer sets for the genes of deubiquitinating enzymes prepared in our laboratory (including the primer sets of Korean Patent Publication No. 10-2018-0050098), in each cDNA diluted to 250 ng. The multiplex RT-PCR was carried out when the amount of amplified GAPDH was constant. The multiplex RT-PCR was performed by adding 2× premix for multiplex RT-PCR (SMP01-M25h, Solgent, Daejeon, Korea) and 12 groups of deubiquitinating enzyme primers for multiplex RT-PCR to each cDNA. The sequences of each primer of the primer sets used for the multiplex RT-PCR are as shown in Tables 1 to 5. PCR conditions were as follows: denaturation step at 95° C. for 20 seconds, annealing step at 60° C. for 40 seconds, and extension step at 72° C. for 60 seconds, for a total of 40 cycles. The above Multiplex RT-PCR analysis was repeated four times.

TABLE 1

| | Deubiquitinating | | | |
|---|---|---|---|---|
| Group | enzyme | Primer | Sequence | Size |
| G1 | USP5 | Forward | GTC CAC AAA GAC GAG TGC GCC T | 409 |
| | | Reverse | AGG CTG AGT CGG CCG ACA GTA | |
| | USP8 | Forward | GGA AAG TAA AAC TGT CCT GCG CAA | 348 |
| | | Reverse | CTA GGC ACT GGC TGA ATT ATG GG | |
| | USP4 | Forward | GTA GAA GGC CAG CAA CCC ATC G | 311 |
| | | Reverse | ACT AGC ACC TGA CCC TGG TAT AG | |
| | USP9X | Forward | GCT TCA AGG GTT CCA GGA CAA G | 263 |
| | | Reverse | GAA GAC TAT CTC GCA ACA CTA TGG | |
| | USP51 | Forward | GGA CCC CAG AGA CTA GGA AAC G | 210 |
| | | Reverse | CAT AAT CCT TAC ACA TGA AGC A | |
| | USP27 | Forward | CTC CAG CTT TAC GAT CGG TTT AAG | 184 |
| | | Reverse | CCG AAA CAG CGA CGA CAT CTC AC | |
| | USP47 | Forward | CGA TGA TCA ACA TGT CAG CAG GA | 150 |
| | | Reverse | TTT CTG GCT GGA TCC TTC AGT CT | |
| | USP42 | Forward | TTA CTC ATC CCA CCC ATA GCC | 120 |
| | | Reverse | TCA TGT GAG AGG GAA GCT GTG GT | |
| | USP45 | Forward | ACT GTC AGT CTC CTT GGT GTA CAG | 100 |
| | | Reverse | TGG GCT GTT CAG ATC CAG TAG T | |
| G2 | USP1 | Forward | GAC CAA ATG TGT GAA ATA GGT AAG C | 493 |
| | | Reverse | GCA AGT AAG GAG TAG AAG TAG GAG | |
| | USP11 | Forward | TGG TGG AAG GCG AGG ATT ATG TG | 410 |
| | | Reverse | GCT GGG CCA AGT GCC ATC TTT C | |
| | USP13 | Forward | ACC CAG CTG GAC AAT GGA GTC A | 351 |
| | | Reverse | CAG CTT GAT GTC ATT GTC CTG GA | |
| | USP12 | Forward | GAA CTC TGA GTC TGG TTA CAT CCT | 301 |
| | | Reverse | GAG GAG CTG GTA TCT CTG ATT TCA | |
| | USP14 | Forward | TCA GTG TAT TCG TTC TGT GCC TGA | 268 |
| | | Reverse | CTC GCT CAT TTG TAT CCA ACA TTCA | |
| | USP15 | Forward | AAA CCT CGC TCC GGA AAG GGG A | 222 |
| | | Reverse | CAG TTG GCA ACA GTA TGT AAT CCA A | |
| | USP54 | Forward | CGT GGT AGT GTA CAA GGG ATG TTT | 181 |
| | | Reverse | CTC CCA TGC ACT TGT GAG TTG TAA | |
| | USP48 | Forward | GCT GGT AGA TCG GGA TAA TTC CA | 150 |
| | | Reverse | AAC TCA TAG GGC TCA GCT CCA G | |
| | USP46 | Forward | CCA ATC CTG CTG ATG TGG CAG TC | 120 |
| | | Reverse | GCT GAT GGC TGG AAA GAT GTA GTA | |
| | USP52 | Forward | TCT GGC AAG GTT TCC CTG AGA GA | 100 |
| | | Reverse | GGT TGC CAT GCA CAT CAA AGT CT | |

TABLE 2

| | Deubiquitinating | | | |
|---|---|---|---|---|
| Group | enzyme | Primer | Sequence | Size |
| G3 | USP10 | Forward | GGA ACA GTT CTG TGT GGC ACA CA | 510 |
| | | Reverse | CGT AAC TGA CGG GGG CAT GTC AC | |
| | USP34 | Forward | CAG CCA TAG TGC TGA AGT TCA AGT | 410 |
| | | Reverse | GAC TGA CAT CAC CAG ATT GTG CT | |
| | USP18 | Forward | ATT GGA CAG ACC TGC TGC CTT A | 350 |
| | | Reverse | AAG GAG TCC TTC ACC CGG ATC G | |
| | USP21 | Forward | TGA CAA AGC CGG AAG TCC TGT A | 250 |
| | | Reverse | AAA GGG CTT CAC AGG TGC CAG A | |
| | USP16 | Forward | AAA CTT TAG AAC CTG TGT GCA G | 210 |
| | | Reverse | CCT GAG AAT TTC TGC CAC AGC C | |
| | USP33 | Forward | CCC TTG GTA CTT GTCA GGA TTG TA | 180 |
| | | Reverse | AAG CAT AAC ACC ATA CTC GAA GAG | |
| | USP53 | Forward | GAC ATT TCC AGA GAA TGT GCT CTG | 149 |
| | | Reverse | GAT CCA GAT TGG AAA TGT GAA AGG | |
| | USP3 | Forward | AGC TTT CAC AGC TGA CAG GCA TAA | 120 |
| | | Reverse | AAA TTC CGA AGG CCT GTG GCA C | |
| | USP19 | Forward | GTT CTT TCC TTC ATC GTC AGG GTC | 100 |
| | | Reverse | AGT GGG AGT AGC CAA GAG ATC ATG | |
| G4 | JOSD1 | Forward | GTG TCT ACT ACA ACC TGG ACT C | 349 |
| | | Reverse | TCC TCC AAC TCT GAT GAG CCT C | |
| | BRCC3 | Forward | TCC TGT GCC AGG AGG AGC AGG AT | 302 |
| | | Reverse | GAT TCT TGG TAA ACA CTG AGC C | |
| | JOSD2 | Forward | GTG TCT ACT ACA ACC TGG ACT C | 257 |
| | | Reverse | ATG AAG TGC TGG CCT TTC CCA G | |
| | EIF3S | Forward | TCC GCC TGG CTT TGC TCT TCC A | 150 |
| | | Reverse | TTG TCG ACA GTT CCC AAC AGG G | |
| | Ataxin3 | Forward | GTC CAA CAG ATG CAT CGA CCA A | 124 |
| | | Reverse | CGT CTA ACA TTC CTG AGC CAT C | |
| | STAMBP | Forward | GAA GCC CTC CTT AGA TGT GTT | 101 |
| | | Reverse | TGT CCA CCA CAG GTG GCT TAG CT | |

TABLE 3

| | Deubiquitinating | | | |
|---|---|---|---|---|
| Group | enzyme | Primer | Sequence | Size |
| G5 | COPS5 | Forward | GCA GTG GTG ATT GAT CCA ACA A | 301 |
| | | Reverse | AGA CCT GAC CAG TGG TAT AGT C | |
| | PRPF8 | Forward | TCT ATG ACG ACT GGC TCA AGA C | 250 |
| | | Reverse | ATC GCC ATG CTT GTT GAC AGT G | |
| | PSMD14 | Forward | GGT TTG ACA CTT CAG GAC TAC A | 200 |
| | | Reverse | GAG GTC ATA AGT ACA TCC ACAT G | |
| | PSMD7 | Forward | ACG TCT TCA ACC TGC TGC CAG A | 181 |
| | | Reverse | TCC TGC CCT TCT TTC TTC TCT G | |
| | COPS6 | Forward | AGG TGT TCA AGG AGC TGG AGT T | 151 |
| | | Reverse | GGA AGA TCT GTG TGC TTG GTC A | |
| | STAMBPL1 | Forward | TTC GAA GAT CAA CTC AAG AAG CA | 118 |
| | | Reverse | TCT GGT GTG TGG AAA AGC AGG A | |
| | EIF3S3 | Forward | GTC CAA ACT CTT CAA ACC ACC A | 100 |
| | | Reverse | AGT GAA CTC CTT GAT GTT CTG G | |
| G6 | ZRANB1 | Forward | CTA GTG CAA GAC CAA GGG TGA A | 301 |
| | | Reverse | ACA CAT CTT TTA GCC TTG GCC C | |
| | OTUB1 | Forward | AGG AAC CTC AGC AGC AGA AGC A | 244 |
| | | Reverse | GTC TTG CGG ATG TAC GAG TAC T | |
| | OTUD1 | Forward | ATG GGG CAG ATG CTG AAT GTG A | 199 |
| | | Reverse | TGC ACC AGT TGT CGT ACT CTG | |
| | TNFAIP3 | Forward | CCG AGC TGT TCC ACT TGT TAA CA | 181 |
| | | Reverse | CAA CTT TGC GGC ATT GAT GAG A | |
| | OTUD5 | Forward | ATC GGA GGA GTC ATG GAT TGA A | 150 |
| | | Reverse | ACC TGG CGA GCC TGT TTC TCC T | |
| | VCPIP1 | Forward | GCT CGC TAT GGA ATG GAC AAA C | 122 |
| | | Reverse | ACA TGC TCT GGT TCT ATG AGG | |
| | OTUB2 | Forward | CAT TCT TCG GGA CCA TCC TGA A | 100 |
| | | Reverse | GTT CCC ATC CCC TTT GGT CTT | |
| G7 | OTUB6B | Forward | AAG AAT GCT GTT CCC AAG AA | 301 |
| | | Reverse | CCA TAT GTC TGG CTC CTG TT | |
| | OTUD7B | Forward | ACT TCA CAG GGG TGC CTT GTT | 206 |
| | | Reverse | TCC TGT TGT TAC AGG GAA GAA C | |
| | OTUD3 | Forward | GAA GAC GAC CTG AGA GAT GA | 188 |
| | | Reverse | CTG GGC TCA AGA TTC TCT TC | |
| | OTUD4 | Forward | GCT CTG CTA TGT GTC AGT CTC T | 155 |
| | | Reverse | TTA CTT GCA ACT GTC ATC CTC TG | |
| | PAPR11 | Forward | CAG CTA CAA GAT AGA CTT TGC AG | 124 |
| | | Reverse | GAT GGC CTC GTT TTC ACA GAT G | |
| | YOD1 | Forward | ACT TGC CCA TCC AAT CTG GTG A | 100 |
| | | Reverse | ACG TAA CTA GAA GCA CCA CGT T | |

TABLE 4

| | Deubiquitinating | | | |
|---|---|---|---|---|
| Group | enzyme | Primer | Sequence | Size |
| G8 | OTUD7A | Forward | GCA GCA CTT CTA CAT GAT CCT A | 404 |
| | | Reverse | TGT GTA GAT TGG CAT CTC CAG G | |
| | USP35 | Forward | AAG TAC ATG CTC CTG ACC TTC CA | 300 |
| | | Reverse | CCC AGG TTG ATG AGA CCA ATC TT | |
| | USP26 | Forward | CAG CCA CCT GTG AGA CCT GGT AA | 200 |
| | | Reverse | CTG ATA ACT CTC CGC AAG TAA G | |
| | USP17 | Forward | GAG CAA CGC AAG GAG AGC TCA AG | 170 |
| | | Reverse | AGG GTA CCT TCG ACT TTT CTG ACG | |
| | USP50 | Forward | CTA TGA TAC CCT TCC AGT TAA GG | 100 |
| | | Reverse | TGG CAT TCA CGC AGC ATG TGT TG | |
| G9 | USP49 | Forward | AGG ACT ACG TGC TCA ATG ATA ACC | 350 |
| | | Reverse | GCA GGA GCA GCC GTG CAC TCT | |
| | ATXN3L | Forward | TCA GAA GAA AGT GAT GAG TCT GG | 332 |
| | | Reverse | CTC TCA ATT GCT CTC GAA CTT G | |
| | USP7 | Forward | CTC TCA GAC CAT GGG ATT TCC AC | 300 |
| | | Reverse | ATC CTG CTC TGT CAC ACA TAA CTG | |
| | USP9Y | Forward | GAG GCT GTG AGT GGC TGG AAG T | 160 |
| | | Reverse | CGG ACG TGT ACC ATT GTA GAT ATG | |
| | USP2 | Forward | TAT GGT GCC TAC ACC CCG TCC T | 100 |
| | | Reverse | TGA GGA AGT TGC TGG TGG GGA C | |
| G10 | USP29 | Forward | GCA ACA TAG CGC TAT GGA GTC TAA | 288 |
| | | Reverse | AGC TTC TTT CAG CTT AGT CAT CCC | |
| | USP41 | Forward | GGT TCT GCT TCA ATG ACT TTA ATA | 250 |
| | | Reverse | CAG CCT TTA AGA TGT CAT ACT GG | |
| | OTUD6A | Forward | TGG ATG ATC CGA AGA GTG AAC | 202 |
| | | Reverse | TCT TGG AAC TTC TCC AGC TCC T | |
| | USP6 | Forward | CGT TGG AAT CAA CAG CAG CAT TGA | 121 |
| | | Reverse | CAT CCA TCC GCT CGT TCG TGT CA | |

TABLE 5

| | Deubiquitinating | | | |
|---|---|---|---|---|
| Group | enzyme | Primer | Sequence | Size |
| G11 | USP38 | Forward | CGT GTT GGG CCT CCT TCA TC | 402 |
| | | Reverse | TGC AGG GAA GGC AGT AGT GT | |
| | USP31 | Forward | TGA GGA TTG GTG TGG CCG TA | 368 |
| | | Reverse | AAT CTT GTC GCT GCC TGC TC | |
| | USP28 | Forward | GAG GCA GCC CCA ACT GAA TC | 242 |
| | | Reverse | TGC TCA GAT GAC AAG CAG CG | |
| | BAP1 | Forward | TCC GTG ATC TGG GTC CTG TC | 195 |
| | | Reverse | TCC CCG TCT TCT CTC TGC TG | |
| | CYLD | Forward | GCC AAG AAA AAG TCA CTT CAC CC | 177 |
| | | Reverse | TGC CTT TTT GCA GAA GGA ATC CT | |
| | USP43 | Forward | GAC AGA GCTGTT TCC TGG GC | 141 |
| | | Reverse | ATA GCT GCA GGC CAC AGA GA | |
| | USP20 | Forward | TGG GCT CCT CTT CCA AGT TCT | 122 |
| | | Reverse | AGG TTT CAG GTC ATC GTC CTC T | |
| | USP22 | Forward | ACC AAC CAA ACG GGA GCT TG | 100 |
| | | Reverse | CCC AAG GTT GAT CAG CCC AC | |
| G12 | USP36 | Forward | TCC CAG ACA CCC ACA CAC AT | 467 |
| | | Reverse | GTG GTG TTG CTC AGG ACA GG | |
| | USP24 | Forward | CCG ACA GTT GTC CGT GTC TG | 380 |
| | | Reverse | TCC GAA GCT GTA GGC ACG TA | |
| | MPND | Forward | CGG GCA GAC CTT CAA CTC AC | 285 |
| | | Reverse | CCC AGT GGT CTC CGA CTC TT | |
| | USP32 | Forward | CCA TGC AGT GGT GGC AAC A | 233 |
| | | Reverse | GCT GCC AGC AGT TTC TGA GG | |
| | USP39 | Forward | GGA GTC TCG CGG TTC CAC T | 192 |
| | | Reverse | CGC ACA AAC GGG ACA ACA GA | |
| | USP37 | Forward | CAG AAG GAA ACC AGC AGG CA | 159 |
| | | Reverse | CGT CCG AGC TAT TCC ACT TCC | |
| | USPL1 | Forward | TCC CAA GTG ACA GAT AAA GAA GCT G | 108 |
| | | Reverse | ACC CAC AGA ACA CGA TGT TAA AGA | |

(5) qRT-PCR

Using cDNA diluted to 100 ng as a template, qRT-PCR was carried out in a StepOne™ Real-Time PCR System (4376357, Thermo Fisher Scientific, Waltham, MA, USA). Using SYBR™ Green PCR Master Mix (4309155, Thermo Fisher Scientific, Waltham, MA, USA), qRT-PCR was performed under the PCR conditions as follows: denaturation step at 95° C. for 15 minutes; denaturation step at 95° C. for 20 seconds, annealing step at 60° C. for 40 seconds, extension step at 72° C. for 1 minute (40 cycles in total) as a cycling step; at 95° C. for 15 seconds, 60° C. for 1 minute, 95° C. for 15 seconds as a melting step. Thereafter, the mRNA expression levels of USP12, USP17, USP46, USP51, OTUD1, and PSMD14 were analyzed with respect to GAPDH, using the 2^{-ΔΔCt} method. The primer sets used for the housekeeping gene GAPDH and the deubiquitinating enzymes are as shown in Table 6.

TABLE 6

| Gene | SEQ ID NO | Sequence | |
|---|---|---|---|
| USP12 | 1 | Forward | 5′-GAA CTC TGA GTC TGG TTA CAT CCT-3′ |
| | 2 | Reverse | 5′-GAG GAG CTG GTA TCT CTG ATT TCA-3′ |
| USP17 | 3 | Forward | 5′-GAG CAA CGC AAG GAG AGC TCA AG-3′ |
| | 4 | Reverse | 5′-AGG GTA CCT TCG ACT TTT CTG ACG-3′ |
| USP46 | 5 | Forward | 5′-CCA ATC CTG CTG ATG TGG CAG TC-3′ |
| | 6 | Reverse | 5′-GCT GAT GGC TGG AAA GAT GTA GTA-3′ |
| USP51 | 7 | Forward | 5′-GGA CCC CAG AGA CTA GGA AAC G-3′ |
| | 8 | Reverse | 5′-CAT AAT CCT TAC ACA TGA AGC A-3′ |
| OTUD1 | 9 | Forward | 5′-ATG GGG CAG ATG CTG AAT GTG A-3′ |
| | 10 | Reverse | 5′-TGC ACC AGT TGT CGT ACT CTG-3′ |
| PSMD14 | 11 | Forward | 5′-GGT TTG ACA CTT CAG GAC TAC A-3′ |
| | 12 | Reverse | 5′-GAG GTC ATA AGT ACA TCC ACAT G-3′ |
| GAPDH | 13 | Forward | 5′-ATC CCA TCA CCA TCT TCC-3′ |
| | 14 | Reverse | 5′-CCA TCA CGC CAC AGT TTG-3′ |

### (6) Verification and Analysis of the Results

The densitometric analysis was performed with Image J (National Institutes of Health, Bethesda, MD, USA) and the Turkey analysis was performed with GraphPad Prism version 5 (GraphPad Software, La Jolla, CA, USA). ANOVA was performed by a one-way analysis to show significant differences.

### 2. Test Results

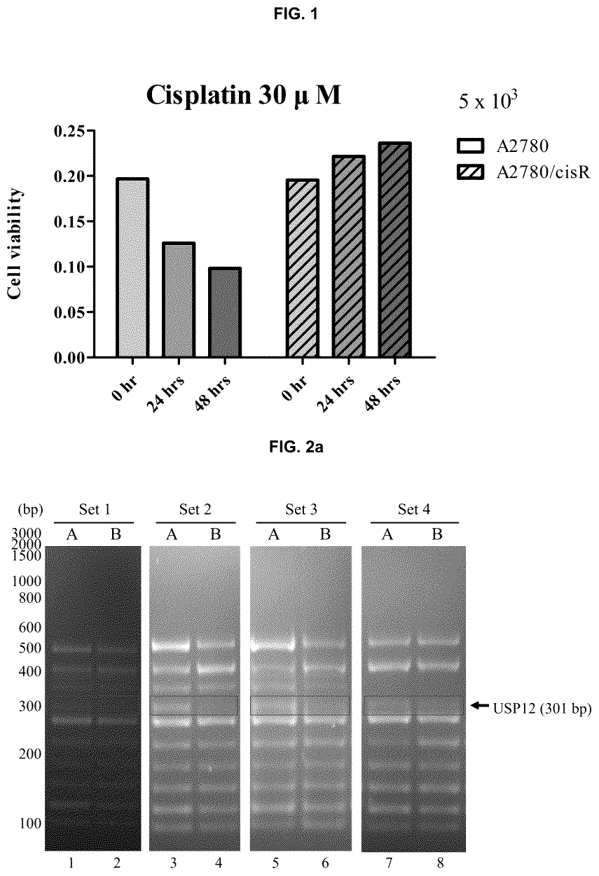

After treating the ovarian cancer cell line A2780 and the cisplatin-resistant ovarian cancer cell line A2780/cisR with cisplatin (30 μM) and then culturing for 0, 24, and 48 hours, the results obtained by measuring the cell viabilities using the CCK-8 kit are shown in FIG. 1. A2780 showed a decrease in cell viability after the treatment with cisplatin, whereas A2780/cisR showed no decrease in cell viability due to resistance to cisplatin and rather showed proliferation.

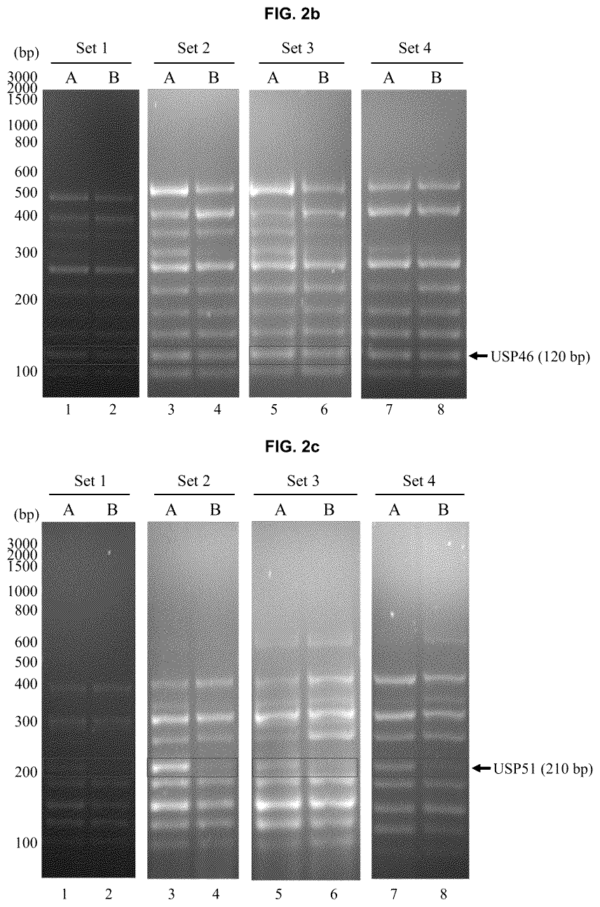

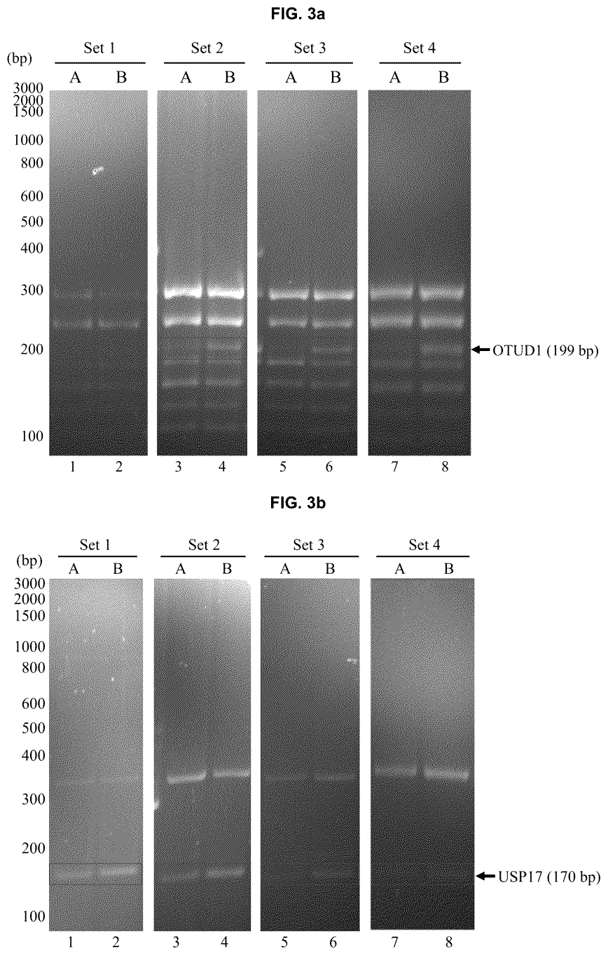

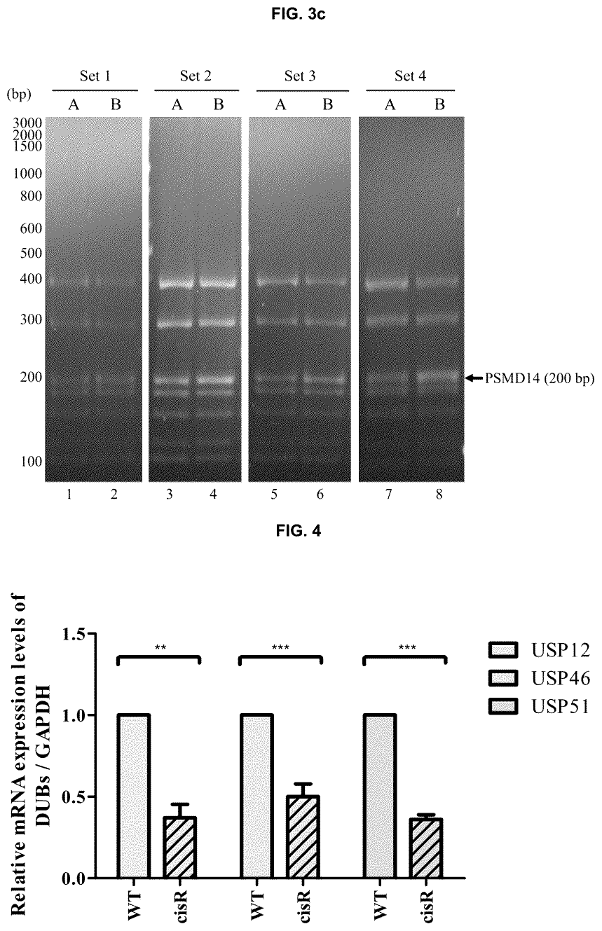

Each RNA was extracted from the ovarian cancer cell line A2780 and the cisplatin-resistant ovarian cancer cell line A2780/cisR, cDNA was synthesized therefrom, the multiplex RT-PCR was performed, and then the results were analyzed by gel electrophoresis (FIGS. 2 and 3). FIGS. 2a to 2c show the results of the multiplex RT-PCR analyses indicating the deubiquitinating enzymes that showed decreased mRNA expression levels in the A2780/cisR cells. FIGS. 3a to 3c show the results of the multiplex RT-PCR analyses indicating the deubiquitinating enzymes that showed increased mRNA expression levels in the A2780/cisR cells. From the results of FIGS. 2 and 3, it can be confirmed that the deubiquitinating enzymes showing mRNA expression changes in A2780 cells and A2780/cisR are USP12, USP17, USP46, USP51, OTUD1, and PSMD14.

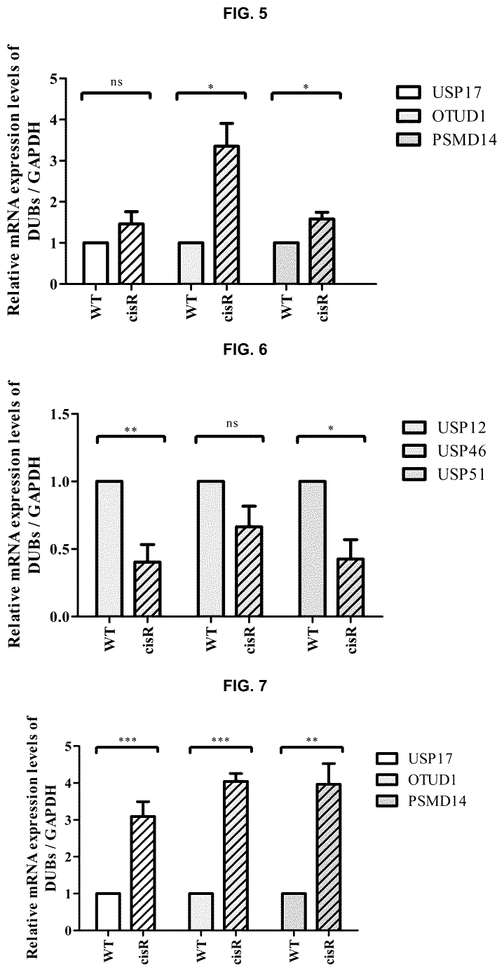

FIG. 4 shows the statistical analysis results of the relative mRNA expression levels of the deubiquitinating enzymes USP12, USP46, and USP51 from the results of FIG. 2. As can be seen from the results of FIG. 4, the expressions of USP12, USP46, and USP51 decreased by 3.11 times, by 2.13 times, and by 2.80 times, respectively. In addition, FIG. 5 shows the statistical analysis results of the relative mRNA expression levels of the deubiquitinating enzymes USP17, OTUD1, and PSMD14 from the results of FIG. 3. As can be seen from the results of FIG. 5, the expressions of USP17, OTUD1, and PSMD14 increased by 1.46 times, by 3.35 times, and by 1.58 times, respectively.

The multiplex PCR results were verified by performing qRT-PCR for confirming the changes in mRNA expression levels of USP12, USP17, USP46, USP51, OTUD1, and PSMD14 in cisplatin-resistant cells (FIGS. 6 and 7). FIG. 6 shows the statistical analysis results of the relative mRNA expression levels of the deubiquitinating enzymes USP12, USP46, and USP51, based on the qRT-PCR results. From the results of FIG. 6, it can be confirmed that the mRNA levels of USP12, USP46, and USP51 decreased by 4.07 times, by 2.03 times, and by 8.83 times, respectively, in the cisplatin resistant cells. FIG. 7 shows the statistical analysis results of the relative mRNA expression levels of the deubiquitinating enzymes USP17, OTUD1, and PSMD14, based on the qRT-PCR results. From the results of FIG. 7, it can be confirmed that the mRNA levels of USP17, OTUD1, and PSMD14 increased by 3.45 times, by 4.04 times, and by 3.97 times, respectively, in the cisplatin resistant cells.

### 3. Discussion

Cisplatin, which is used as an anticancer agent for treating various cancers, is a platinum-based anticancer agent primarily used for treating patients having ovarian cancer, one of the most common gynecological tumors worldwide. Although it shows good efficacy initially, there is a problem that most patients ultimately develop resistance, leading to recurrence and poor prognosis. The present inventors confirmed decreased expressions of the genes encoding the deubiquitinating enzyme USP12, USP46, and USP51 and increased expressions of the genes encoding the deubiquitinating enzyme USP17, OTUD1, and PSMD14, in the ovarian cancer cells A2780/cisR having resistance, through multiplex RT-PCR and qRT-PCR. Previously, difference in the expression of USP46 was identified in the study performed on the cisplatin-resistant ovarian-cancer cells. Therefore, it is possible to construct a model capable of diagnosing cisplatin resistance based on the above five deubiquitinating enzymes, i.e., USP12, USP17, USP51, OTUD1, and PSMD14, in addition to USP46. And, based on the results of this study, it is possible to conduct studies on the deubiquitinating enzymes according to cisplatin resistance and the mechanism of cisplatin resistance. In addition, the genes encoding the deubiquitinating enzymes, i.e., USP12, USP17, USP51, OTUD1, and PSMD14 can be used as a biomarker for diagnosing the resistance.

1. An analytical method for providing information necessary for diagnosing an ovarian cancer patient resistant to cisplatin, comprising measuring an expression level of a gene encoding at least one deubiquitinating enzyme selected from the group consisting of USP12, USP17, USP51, OTUD1, and PSMD14 in a tumor cell sample externally discharged from an ovarian cancer patient.

2. The analytical method according to claim 1, wherein the measuring an expression level of the gene encoding the deubiquitinating enzyme is carried out by measuring an amount of mRNA of the gene.

3. The analytical method according to claim 2, wherein the measuring an amount of mRNA is carried out by RT-PCR or qRT-PCR.

4. The analytical method according to claim 1, comprising measuring an expression level of the gene encoding at least one deubiquitinating enzyme selected from the group consisting of USP12 and USP51.

5. The analytical method according to claim 4, wherein the measuring an expression level of the gene is carried out by measuring an amount of mRNA of the gene with a primer set of SEQ ID NOs: 1 and 2 or a primer set of SEQ ID NOs: 7 and 8.

6. The analytical method according to claim 1, comprising measuring an expression level of the gene encoding at least one deubiquitinating enzyme selected from the group consisting of USP17, OTUD1, and PSMD14.

7. The analytical method according to claim 6, wherein the measuring an expression level of the gene is carried out by measuring an amount of mRNA of the gene with a primer set of SEQ ID NOs: 3 and 4, a primer set of SEQ ID NOs: 9 and 10, or a primer set of SEQ ID NOs: 11 and 12.

8. A kit for diagnosing an ovarian cancer patient resistant to cisplatin, comprising a molecule capable of measuring an expression level of a gene encoding at least one deubiquitinating enzyme selected from the group consisting of USP12, USP17, USP51, OTUD1, and PSMD14, wherein the molecule is a primer having a complementary sequence specific to the gene encoding the deubiquitinating enzyme.

9. The kit according to claim 8, wherein the primer has at least one base sequence selected from the group consisting of SEQ ID NOs: 1 to 4 and 7 to 12.

10. The kit according to claim 8, in the form of a microarray in which the primer is immobilized on a substrate.


SEQUENCE LISTING (14 sequences)

SEQ ID NO: 1 - DNA, 24 bp, synthetic construct
  source 1..24: mol_type = other DNA
gaactctgagtctggttacatcct

SEQ ID NO: 2 - DNA, 24 bp, synthetic construct
  source 1..24: mol_type = other DNA
gaggagctggtatctctgatttca

SEQ ID NO: 3 - DNA, 23 bp, synthetic construct
  source 1..23: mol_type = other DNA
gagcaacgcaaggagagctcaag

SEQ ID NO: 4 - DNA, 24 bp, synthetic construct
  source 1..24: mol_type = other DNA
agggtaccttcgacttttctgacg

SEQ ID NO: 5 - DNA, 23 bp, synthetic construct
  source 1..23: mol_type = other DNA
ccaatcctgctgatgtggcagtc

SEQ ID NO: 6 - DNA, 24 bp, synthetic construct
  source 1..24: mol_type = other DNA
gctgatggctggaaagatgtagta

SEQ ID NO: 7 - DNA, 22 bp, synthetic construct
  source 1..22: mol_type = other DNA
ggaccccagagactaggaaacg

SEQ ID NO: 8 - DNA, 22 bp, synthetic construct
  source 1..22: mol_type = other DNA
cataatccttacacatgaagca

SEQ ID NO: 9 - DNA, 22 bp, synthetic construct
  source 1..22: mol_type = other DNA
atggggcagatgctgaatgtga

SEQ ID NO: 10 - DNA, 21 bp, synthetic construct
  source 1..21: mol_type = other DNA
tgcaccagttgtcgtactctg

SEQ ID NO: 11 - DNA, 22 bp, synthetic construct
  source 1..22: mol_type = other DNA
ggtttgacacttcaggactaca

SEQ ID NO: 12 - DNA, 23 bp, synthetic construct
  source 1..23: mol_type = other DNA
gaggtcataagtacatccacatg

SEQ ID NO: 13 - DNA, 18 bp, synthetic construct
  source 1..18: mol_type = other DNA
atcccatcaccatcttcc

SEQ ID NO: 14 - DNA, 18 bp, synthetic construct
  source 1..18: mol_type = other DNA
ccatcacgccacagtttg